(19) United States
(12) Patent Application Publication
(10) Pub. No.: US 2020/0406007 A1
(43) Pub. Date: Dec. 31, 2020

(54) Catheter System

(71) Applicant: JOLINE GMBH & CO. KG, HECHINGEN (DE)

(72) Inventor: Dieter Seidenberger, Hechingen (DE)

(73) Assignee: JOLINE GMBH & CO. KG, HECHINGEN (DE)

(21) Appl. No.: 16/962,554

(22) PCT Filed: Jan. 24, 2019
(86) PCT No.: PCT/EP2019/051691

§ 371 Date: Jul. 16, 2020

(30) Foreign Application Priority Data
Feb. 20, 2018 (DE) 10 2018 103 742.2

(51) Int. Cl. A61M 25/01; A61M 25/00

(52) U.S. Cl. CPC A61M 25/0108; A61M 25/003; A61M 2025/0031; A61M 25/0017; A61M 25/0102; A61M 25/0071

(57) ABSTRACT

Catheter system for inserting and positioning a double-lumen catheter in a blood vessel, including a double-lumen catheter having a distal catheter tip, with a first lumen and a second lumen, a first mandrin which extends through the first lumen and a second mandrin which extends through the second lumen, both mandrins of the catheter being radio-opaque. The distal end of the second mandrin is arranged at a distance (X) in the proximal direction from the distal catheter tip, the distance between the distal catheter tip and the distal end of the second mandrin corresponding in particular to a functional catheter tip length.

The invention relates to a catheter system for inserting and positioning a double-lumen catheter, in particular a dialysis catheter, in a blood vessel. The catheter system comprises a double-lumen catheter having a distal catheter tip, with a first lumen and a second lumen. The catheter system further comprises a first mandrin which extends through the first lumen and a second mandrin which extends through the second lumen.

Such catheter systems are known from the prior art. Mandrins (also called stylets) are tools for inserting catheters into blood vessels. In the case of double-lumen catheters, as are used in particular in dialysis, mandrins are inserted through both lumens. Mandrins are used to stabilize the catheter against buckling and thus to allow safe insertion of the catheter into a blood vessel. Furthermore, mandrins in the lumens prevent air from penetrating into the blood vessel and prevent undesired leakage and/or penetration of blood from or into the catheter when it is inserted into the blood vessel. US 2016/0008573 A1 discloses catheter systems in which two separate mandrins merge in the distal direction behind the distal end of the catheter tip. US 2006/0058737 A1 discloses a mandrin which is radio-opaque.

A catheter system is known from US 2011/0144620 A1. In this case, an X-ray marker designed as a ring is provided proximal to the most proximal opening in the distal end region of the double-lumen catheter. This X-ray marker can be a ring made of precious metal or a print with tungsten-containing printing ink. These X-ray markers are used to correctly implant the catheter in the blood vessel. The desired position can be, for example, at the transition from the superior vena cava (SVC) to the right ventricle (right atrium (RA)). If the X-ray marker is therefore in this position, the functional region of the catheter protrudes into the right ventricle. The functional region is represented here as the region between the distal catheter tip and the most proximal lateral opening.

The use of such a radio-opaque X-ray marker, which can be recognized by an X-ray device, can be disadvantageous since the radio-opaque material touches the body and has body contact. Furthermore, such conventional X-ray markers can only make small regions visible, specifically the region of the X-ray marker ring. The radio-opaque materials used may in particular not be very body-friendly. In particular in the case of long-term catheters, which remain in the body of a person for several days, weeks or even months, the permanent contact between the X-ray materials and the human body can be disadvantageous.

In the following, the term dialysis is understood to mean all blood purification methods, in particular hemodialysis, hemofiltration, hemodiafiltration, peritoneal dialysis, and hemoperfusion.

The overall problem addressed by the invention is that of providing a catheter system which is more compatible with the body and in particular is rich in contrast and clearly visible.

This problem is solved by a catheter system having the features of claim 1. Thus, according to the invention, both mandrins of the catheter are radio-opaque, with the distal end of the second mandrin being arranged at a distance in the proximal direction from the distal catheter tip. The distance between the distal catheter tip and the distal end of the second mandrin can correspond in particular to a functional catheter tip length.

Because the mandrins are radio-opaque, they can be recognized by an X-ray device. This is used for the correct positioning of the catheter, specifically in that the second mandrin is arranged at a distance from the distal catheter tip. When the distal end of the second mandrin has been detected at the intended position, the catheter is correctly positioned. The intended position can be the right atrium, for example. From this intended position, the catheter tip then protrudes further into the blood vessel in the distal direction. This region between the distal end of the second mandrin and the distal catheter tip can correspond in particular to a functional catheter tip length which protrudes into the blood vessel, for example into the right atrium, starting from the intended position. The distal end of the second mandrin can be located, for example, at the most proximal opening of the second lumen. The functional portion is therefore located, proceeding from this most proximal opening, in the distal direction. The first mandrin of the first lumen can be located on the distal catheter tip, or even project beyond it, such that buckling of the catheter tip can be prevented, in particular when the catheter system is inserted into a blood vessel.

The second lumen has a distal opening, with at least one lateral opening being present in the proximal direction, and the distal end of the second mandrin being located behind a proximal end of the most proximal lateral opening in the proximal direction. Alternatively, the distal end of the second mandrin can be located at the proximal end of the most proximal lateral opening. Consequently, in particular the distance between the distal catheter tip and the distal end of the second mandrin can represent the functional catheter tip portion. Lateral openings can be used in particular so that, when the distal lumen opening is closed, for example by abutting a blood vessel, blood can nevertheless be introduced or withdrawn through the catheter.

The invention makes it possible to completely dispense with an X-ray marker provided on the outside of the catheter, which can have harmful effects on the human body. Instead, the mandrins are used for catheter positioning. These are removed after the positioning of the catheter in order to use the catheter, for example for dialysis. Overall, a catheter system that is more compatible with the body can be provided. An image of the entire distal catheter tip geometry and position can also be obtained.

According to an advantageous development of the invention, the distance between the catheter tip and the distal end of the second mandrin is between 15 and 50 mm, in particular between 15 and 30, more particularly 20 mm. This region can correspond in particular to the functional catheter tip length and can in particular represent the region between the most proximal opening of the second lumen and the distal tip of the catheter.

According to a further advantageous embodiment of the invention, the distal end of the second mandrin is located within the second lumen. Consequently, the distal end of the second mandrin can in particular also be spaced apart from a distal opening of the second lumen. The first lumen can correspond in particular to the venous lumen of a dialysis catheter, and therefore the first mandrin is the venous mandrin. This venous lumen allows purified blood to be supplied to the blood vessel in the later use of the catheter. Accordingly, the second lumen can be the arterial lumen and the second mandrin can be the arterial mandrin. Blood can be withdrawn from the blood vessel through this arterial lumen and supplied to the dialyzer for purification.

It is particularly preferred if the first mandrin projects beyond the catheter tip in the distal direction for insertion of the catheter. The first mandrin can therefore be used as a guide mandrin so that the catheter can be placed securely. Because the first mandrin is also radio-opaque, the position of the first mandrin can be determined and, in particular, its distal end can be visualized by means of an X-ray device, in particular to prevent blood vessels from being damaged when the catheter is inserted.

It is particularly preferred if the first mandrin and/or the second mandrin has/have a BaSO4 content between 15-30 wt. %, in particular between 20 and 25 wt. %, more particularly 24.5 wt. %. Such a barium sulfate content allows an X-ray opacity to be provided so that the material is visible in the X-ray contrast. The mandrins can in particular be designed as profile tubes. In addition to the BaSO4 content mentioned, the mandrins can comprise a mixture (compound) of high-density polyethylene (HDPE) and low-density polyethylene (LDPE). For example, the HDPE content can be approximately 34 wt. %, while the LDPE content can be approximately 39 wt. %.

The two-lumen catheter can consist of a polyurethane (PUR), for example a thermoplastic polyurethane elastomer (TPU). The catheter can also have a BaSO4 content. 20 wt. % BaSO4 would be conceivable. The catheter can have a Shore hardness of 85 A.

It is furthermore particularly preferred if the first lumen comprises a distal opening, the region of the catheter surrounding the opening comprising the catheter tip. The distal opening of the first lumen can thus be located behind the distal opening of the second lumen in the distal direction, the distal opening of the second lumen therefore being proximal to the distal opening of the first lumen. Such an embodiment of the catheter can allow an optimal withdrawal of blood and an optimal introduction of purified blood into the blood vessel, in particular in the case of dialysis catheters.

The catheter preferably has a central wall that separates the first lumen from the second lumen, the distal end of the central wall representing the catheter tip. Such a catheter has a particularly simple design. It can therefore have, in particular, two tube portions which extend in parallel with one another and each form at least one lumen, the central wall being located between the two tube portions.

A particularly preferred development of the invention results from the fact that the catheter is designed as a dialysis catheter, the first lumen being the venous lumen and the second lumen being the arterial lumen.

The catheter is preferably designed for long-term implantation. Such a catheter can remain in the patient in particular for several days, several weeks or several months or up to a year. The invention is particularly advantageous in such long-term applications because an X-ray marker, which can contain tungsten, for example, and is in contact with the body, can be omitted and the catheter is instead positioned by means of the mandrin. Such a catheter can have fixation means in particular in the proximal region in order to fix the catheter position.

Further details and advantageous embodiments of the invention can be found in the following description, on the basis of which the embodiment of the invention shown in the figures is described and explained in more detail. In the drawings:

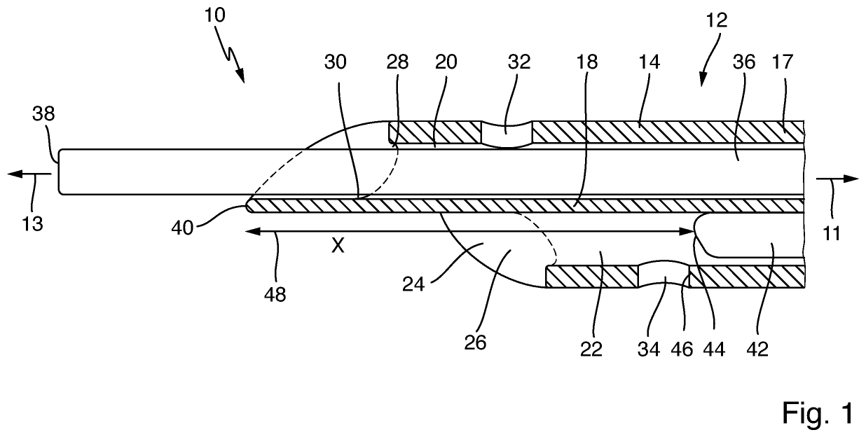

FIG. 1 is a schematic longitudinal sectional view of the distal end region of a catheter system according to one embodiment.

FIG. 1 shows overall the distal end region 12 of a catheter system 10, firstly comprising a catheter 14 designed as a dialysis catheter, which is designed for insertion into a blood vessel.

The proximal region of the catheter system 10 is not shown. The proximal region (not shown) of the catheter 14 is designed for connection to a dialyzer via a blood tube system, in order to purify withdrawn blood and supply purified blood to a patient. The proximal end region of the catheter 10 can in particular be designed as in one of the embodiments disclosed in DE 10 2017 118 820.7, the disclosure of which is fully incorporated into the present application by reference.

The catheter 14 has a round contour in cross section with an outer wall 17. Furthermore, the catheter 14 has a central wall 18 which extends along a longitudinal axis and forms a central plane. The central wall 18 separates the inside of the catheter into a first lumen 20, which is designed as an introduction lumen (venous lumen), and a second lumen 22, which is designed as a withdrawal lumen (arterial lumen). Blood can be withdrawn from a patient through the withdrawal lumen 22, purified in the dialyzer, and the blood purified in this way can then be returned to the patient through the introduction lumen 20. At its distal end 24, the withdrawal lumen 22 has a distal opening 26 designed as a withdrawal opening. The introduction lumen 20 has, at its distal end 28, a distal opening 30 designed as an introduction opening.

As can further be seen from FIG. 1, the introduction lumen 20 has a venous lateral opening 32 spaced apart from the introduction opening 30 in the proximal direction 11. This lateral opening 32 is used to ensure that blood can be introduced even if the introduction opening 30 were to be closed, for example by abutting a blood vessel. Accordingly, as is clear from FIG. 2, an arterial lateral opening 34 spaced apart from the withdrawal opening 26 in the proximal direction 11 is provided on the withdrawal lumen 22, so that blood can be withdrawn even if the withdrawal opening 26 were to be closed, for example by abutting a blood vessel. The introduction opening 28 is therefore located behind the withdrawal opening 26 in the distal direction 13, so that the introduction opening 28 is more distal than the withdrawal opening 26.

The exact geometry of the distal end region of the catheter 14, shown schematically in FIG. 1, can in particular be designed as in one of the embodiments disclosed in DE 10 2017 118 819.3, the disclosure of which is fully incorporated into the present application by reference.

A first mandrin 36 is arranged in the introduction lumen 20. This therefore extends through the introduction lumen 20. This is the venous mandrin. The distal end 38 of the first mandrin 36 protrudes in the distal direction 13 beyond the distal end 40 of the catheter 14. The distal end 38 consequently forms the distal end of the catheter system 10.

A second mandrin 42 is also arranged in the withdrawal lumen 22. The distal end 44 of the second mandrin 42 ends at the proximal end 46 of the arterial lateral opening 34. The distance X between the distal end 44 and the catheter tip 40 represents the functional catheter tip portion 48, which consequently extends from the proximal end 46 of the lateral opening 34 to the catheter tip 40. This distance X can be 20 mm, for example.

Both mandrins 36 and 42 are radio-opaque. These contain, for example, 24% barium sulfate (BaSO4), so that the mandrins consist of material that is visible in the X-ray contrast and can be recognized by an X-ray device. Thus, when the catheter is inserted into a blood vessel, the position, in particular the distal end 38 of the first mandrin 36, can first be determined in order to thus supply the catheter into the blood vessel in the distal direction 13 to its intended position.

The intended position can be, for example, at the transition between the superior vena cava (SVC) and the right ventricle (right atrium (RA)). This intended position can in particular be achieved when the distal end 44 of the second mandrin 42 has reached this position. The functional portion 48 of the catheter 14 thus protrudes further from this intended position in the distal direction 13 into the blood vessel, for example the right atrium. The surgeon can thus determine in particular where the proximal end 46 of the most proximal lateral opening 34 in the withdrawal lumen 22 is located. After positioning, the mandrins 36 and 42 are removed. Dialysis can then be carried out, with blood being withdrawn from the blood vessel through the withdrawal lumen 22 and supplied to a dialyzer for purification, and the purified blood being returned to the blood vessel via the introduction lumen 20. The catheter 14 can in particular be a long-term catheter, which can remain in the body in particular for several days, weeks or months or up to a year.

In contrast with the prior art, an X-ray marker, in particular containing tungsten, on the outside of the catheter in the region of the proximal end 46 of the most proximal lateral opening 34 can be completely dispensed with. Only the catheter 14 remains on the body in contact therewith.

1. Catheter system for inserting and positioning a double-lumen catheter in a blood vessel, comprising a double-lumen catheter having a distal catheter tip, with a first lumen and a second lumen, a first mandrin which extends through the first lumen and a second mandrin which extends through the second lumen, the second lumen having a distal opening and, in the proximal direction, at least one lateral opening, characterized in that both mandrins of the catheter are radio-opaque, the catheter being designed for insertion into the blood vessel such that the distal end of the second mandrin is arranged behind, in the proximal direction, or at a proximal end of the most proximal lateral opening and at a distance (X) in the proximal direction from the distal catheter tip, the distance (X) between the distal catheter tip and the distal end of the second mandrin representing a functional catheter tip portion.

2. Catheter system according to claim 1, wherein the distance between the distal catheter tip and the distal end of the second mandrin is between 15 and 50 mm.

3. Catheter system according to claim 1, wherein the distal end of the second mandrin is located within the second lumen.

4. (canceled)

5. Catheter system according to claim 1, wherein the first mandrin projects beyond the catheter tip in the distal direction for insertion of the catheter.

6. Catheter system according to claim 1, wherein at least one of the first mandrin and/or the second mandrin have a BaSO4 content between 15-30 wt. %.

7. Catheter system according to claim 1, wherein the first lumen comprises a distal opening, and wherein the region of the catheter surrounding the opening comprises the catheter tip.

8. Catheter system according to claim 1, wherein the catheter has a central wall that separates the first lumen from the second lumen, and wherein the distal end of the central wall represents the catheter tip.

9. Catheter system according to claim 1, wherein the catheter is designed as a dialysis catheter, wherein the first lumen is the venous lumen, and wherein the second lumen is the arterial lumen.

10. Catheter system according to claim 1, wherein the catheter is designed for long-term implantation.

11. A method for inserting and positioning a double-lumen catheter in a blood vessel, comprising the steps of:
a. using a double-lumen catheter having a distal catheter tip, with a first lumen and a second lumen, a first radio-opaque mandrin which extends through the first lumen and a second radio-opaque mandrin which extends through the second lumen, the second lumen having a distal opening and, in the proximal direction, at least one lateral opening, and
b. inserting the catheter into the blood vessel such that the distal end of the second mandrin is arranged behind, in the proximal direction, or at a proximal end of the most proximal lateral opening and at a distance in the proximal direction from the distal catheter tip, wherein the distance between the distal catheter tip and the distal end of the second mandrin represents a functional catheter tip portion.
